(19) United States
(12) Patent Application Publication
(10) Pub. No.: US 2011/0015587 A1
(43) Pub. Date: Jan. 20, 2011

(54) Irrigation Device and Method Using Same

(76) Inventors: David M. Tumey, Germantown, MD (US); Pal (Paul) Svedman, Malmo (SE); Richard M. Vogel, Gaithersburg, MD (US); Mark S. Meents, Germantown, MD (US)

Correspondence Address: A PATENT LAWYER CORP, PLC;R WILLIAM GRAHAM, 22 S ST CLAIR ST, DAYTON, OH, 45402, US

(21) Appl. No.: 12/787,465

(22) Filed: May 26, 2010

Related U.S. Application Data
(63) Continuation-in-part of application No. 12/502,740, filed on Jul. 14, 2009.

(51) Int. Cl. A61M 1/00

(52) U.S. Cl. 604/290; 604/319

(57) ABSTRACT

A disposable therapeutic device for the promotion of wound healing providing fluid irrigation and vacuum drainage of a wound includes a housing containing a controller and fluid moving device in a waterproof manner, a fluid mover capable of raising, compressing, or transferring fluid, a controller equipped to restrict fluid moving device in accordance with a predetermined treatment plan or duration, a chargeable power source removably connected to the housing, an optional therapeutic member of a compressible dressing or inflatable cuff to provide hemostasis, an identification member for regulating the operation of the device in accordance with a predetermined treatment plan, a disposable container, a pressure sensor and a control display panel. The fluid includes, but is not limited to, Lactoferrin, Xylitol, Dakins Solution, Polyhexanide and Hypochlorous Acid.

This is a continuation-in-part of Ser. No. 12/502,740 filed Jul. 14, 2009.

### BACKGROUND

1. Field of Invention

The invention is generally directed to a therapeutic device for the promotion of wound healing. More particularly, the present invention relates to providing fluid irrigation and vacuum drainage of a wound and methods employing the same.

2. Related Art

These devices are normally used in clinical settings such as hospitals or extended care facilities, but patients can often be located in non-clinical environments, where portability, ease of use, and control of therapy parameters is necessary. Such places can, for example, include the home, office or motor vehicles, and at the extreme, military battlefields and other locations where electrical power may be unreliable or unavailable.

Negative pressure wound therapy (NPWT), also known as vacuum drainage or closed-suction drainage, is known. A vacuum source is connected to a semi-occluded or occluded therapeutic member, such as a compressible wound dressing. Various porous dressings comprising gauze, felts, foams, beads and/or fibers can be used in conjunction with an occlusive semi-permeable cover and a controlled vacuum source. In addition to negative pressure, there exist pump devices configured to supply positive pressure to another therapeutic member, such as an inflatable cuff for various medical therapies.

In addition to using negative pressure wound therapy, many devices employ concomitant wound irrigation. For example, a known wound healing apparatus includes a porous dressing made of polyurethane foam placed adjacent a wound and covered by a semi-permeable and flexible plastic sheet. The dressing further includes fluid supply and fluid drainage connections in communication with the cavity formed by the cover, foam and skin. The fluid supply is connected to a fluid source that can include an aqueous topical anesthetic or antibiotic solution, isotonic saline, or other medicaments for use in providing therapy to the wound. The fluid drainage can be connected to a vacuum source where fluid can be removed from the cavity and subatmospheric pressures can be maintained inside the cavity. The wound irrigation apparatus, although able to provide efficacious therapy, is somewhat cumbersome, difficult to use without trained professional medical personnel, and generally impractical outside the clinical setting.

Some devices use vacuum sealing of wound dressings consisting of polyvinyl alcohol foam cut to size and stapled to the margins of the wound. Such dressings are covered by a semi-permeable membrane while suction and fluid connections are provided by small plastic tubes which are introduced into the foam generally through the patient's skin. Such devices alternate in time between vacuum drainage and the introduction of aqueous medicaments to the wound site, but do not do both simultaneously. While the prior devices have proven to be useful in fixed therapeutic sites, such devices require improvement to render broader and greater therapeutic use.

### SUMMARY OF THE INVENTION

It is an object to improve wound healing.

It is another object to improve devices for use in treating wounds.

It is an object to improve a pump for use in treating wounds.

It is yet another object to provide a therapeutic device for treating wounds which has improved method of treatment.

It is yet another object to provide a therapeutic device for treating wounds which is equipped for predetermined and/or remote control of therapy parameters of irrigation, time and pressure.

One embodiment of the invention is directed to a disposable therapeutic device, which includes a fluid mover for one of raising, compressing, or transferring fluid, a therapeutic member operably connected to the fluid mover and actuated thereby, the therapeutic member operably disposably used on a patient in a manner to deliver therapy to the patient as function of actuation of the fluid mover; and controller operably associated with the fluid mover for controlling operation thereof in a manner to cause one of continuous and intermittent actuation of said fluid mover. In a preferred embodiment, there is provided an irrigation fluid in fluid communication with the therapeutic member. The controller is equipped to control the fluid mover in a manner to provide one of continuous irrigation with continuous compression, continuous irrigation with intermittent compression, intermittent irrigation with continuous compression and intermittent irrigation with intermittent compression.

One method employs the device and includes continuous irrigation, continuous compression (NPWT). In this method, the suction is set to operate continuously at a pre-determined pressure level while an irrigation solution, comprised of one or more of a topical treatment irrigation solution which is introduced continuously at a pre-determined rate.

Another method employs the device and includes continuous irrigation with intermittent compression (NPWT). In this method, the suction is set to operate intermittently at predetermined pressure and “on/off” cycle durations while an irrigation solution, comprised of one or more topical treatment solutions, is introduced continuously at a pre-determined rate.

Yet another method employs the device and includes compression (NPWT) with intermittent irrigation (flush before and after dressing change). In this method, the suction is set to operate continuously at a pre-determined pressure level while an irrigation solution, comprised of one topical treatment solutions is introduced intermittently at a pre-determined rate and frequency.

Still another method employs the device and includes intermittent compression (NPWT) with intermittent irrigation (flood and dwell). In this method, the suction is set to operate intermittently at predetermined pressure and “on/off” cycle durations while an irrigation solution, comprised of one or more of the above listed topical treatment solutions, and is introduced intermittently at a pre-determined rate and frequency. The frequency of irrigation fluid introduction is synchronized with the application and non-application of suction pressure.

Optionally, the controller is equipped to restrict use of the fluid mover by the patient in accordance with a predetermined treatment plan or duration and render the pump inoperable. A chargeable power source to supply power to the fluid mover and the controller is provided.

More particularly, a wound irrigation system can use a fluid mover, such as a diaphragm or piston-type pump, to raise, compress and transfer fluid in an electromechanical vacuum apparatus that includes a controller, such as a microprocessor-based device, having stored thereon software configured to control the electromechanical vacuum apparatus, and including one of a timer, for a remote controller of the system, and restriction device for restricting the operation of the apparatus to a predetermined treatment plan or duration.

A first vacuum pump can be electrically associated with the microcontroller and capable of generating a vacuum. An optional second vacuum pump is electrically associated with the microcontroller and is capable of maintaining a predetermined vacuum level. A first electronic vacuum-pressure sensor can be operably associated with the vacuum pump(s) and the microcontroller for monitoring vacuum level.

A fluid-tight wound exudate collection canister can be provided and can include an integrated barrier, such as a float valve, porous polymer filter or hydrophobic filter, to prevent contents from escaping the canister. Single-lumen tubing can be associated with the canister and vacuum pump(s) for communicating vacuum pressure therefrom. A second electronic vacuum-pressure sensor can be operably associated with the canister and the microcontroller for monitoring canister vacuum.

A dressing includes a porous material and semi-permeable flexible cover. Single-lumen tubing is associated with the dressing and the canister to communicate vacuum pressure therefrom. An irrigation vessel can be provided to contain topical irrigation fluid to be used in irrigating the wound. Single-lumen tubing is associated with the irrigation vessel and the dressing to communicate fluid thereto.

The electromechanical vacuum apparatus housing may incorporate a compartment that can hold the irrigation vessel. The electromechanical vacuum apparatus can preferably include a device for regulating the quantity of fluid flowing from the irrigation vessel to the dressing. This device can comprise a mechanical or pneumatically actuated valve or clamp.

The electromechanical vacuum apparatus may include commercially available disposable storage batteries enabling portable operation thereof. Alternative power sources include rechargeable or reprocessable batteries which are removably connected to a housing, which contains the fluid mover and controller, both of which require power in a waterproof environment. Other alternative power sources are solar energy, a manually operated generator in combination with a storage device such as a supercapacitor, or a pneumatic accumulator.

An embodiment of the invention can include a method for improving the generation and control of a therapeutic vacuum. In this embodiment, a multi-modal algorithm monitors pressure signals from a first electronic vacuum-pressure sensor associated with a vacuum pump and capable of measuring the output pressure from the pump. The algorithm further monitors pressure signals from a second electronic vacuum-pressure sensor associated with a collection canister and capable of measuring the subatmospheric pressure inside the canister. The second electronic vacuum-pressure sensor may also be associated with the wound dressing and capable of measuring the subatmospheric pressure inside the dressing. The canister is connected to the vacuum pump by a single-lumen tube that communicates subatmospheric pressure therefrom. The canister is connected to a suitable dressing by a single-lumen tube that communicates subatmospheric pressure thereto.

At the start of therapy, both the first and second electronic vacuum-pressure sensors indicate the system is equilibrated at atmospheric pressure. A first-mode control algorithm is employed to rapidly remove the air in the canister and dressing, and thus create a vacuum. The first-mode implemented by the control algorithm is subsequently referred to herein as the “draw down” mode. Once the subatmospheric pressure in the canister and dressing have reached a preset threshold as indicated by the first and second electronic vacuum-pressure sensors respectively, the algorithm employs a second-mode that maintains the desired level of subatmospheric pressure in both the canister and the dressing for the duration of the therapy. The second-mode implemented by the control algorithm is subsequently referred to herein as the “maintenance” mode.

The second-mode control algorithm is configured to operate the vacuum pump at a reduced speed thus minimizing unwanted mechanical noise. In an alternative embodiment, a second vacuum pump can be used for the maintenance mode, which has a reduced capacity, is smaller, and produces significantly lower levels of unwanted mechanical noise. The second-mode control algorithm is configured to permit the maintenance of vacuum in the presence of small leaks, which invariably occur at the various system interfaces and connection points. The method can be performed by, for example, a microprocessor-based device in accordance with a predetermined regimen to achieve one of the desired treatments as described.

The controller can be provided with a timer for restricting the use as a function of a predetermined time. Alternatively, an identification member can be provided with the device such that the controller restricts use as a function of the identification member. The controller may include a Radio Frequency Identification Chip (RFID) chip available under the trademark Omni-ID™. The controller can be operably associated with a remote control for restricting the use of the device.

### BRIEF DESCRIPTION OF THE DRAWINGS

FIG. 1 is a schematic illustrating the device of the invention.

FIG. 1A depicts a part of the invention.

### DETAILED DESCRIPTION

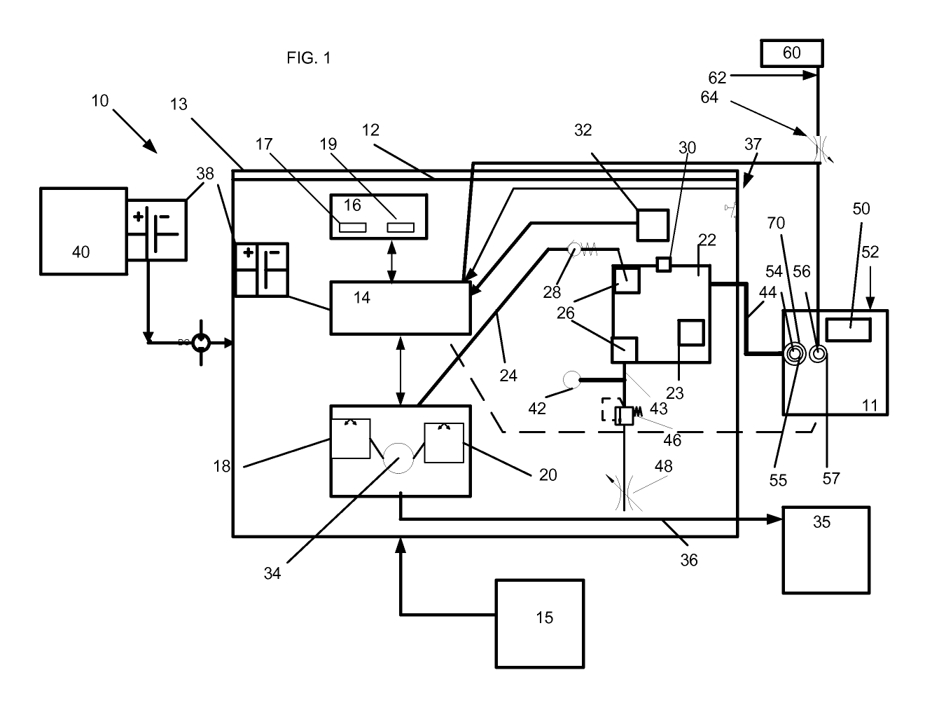

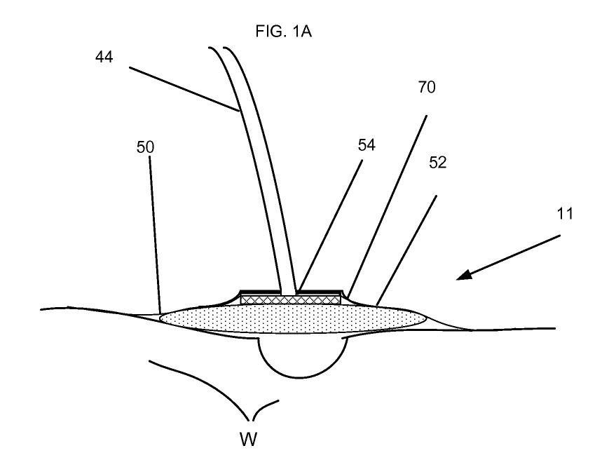

As illustrated in FIG. 1, a disposable therapeutic device of the instant invention is generally designated by the numeral 10. The disposable therapeutic device 10 can preferably include a housing 12 which provides an improved therapeutic device with multiple uses as well as a feature of portability in some applications. The housing 12 can preferably be formed in a waterproof manner to protect components therein. In this regard, housing 12 can have a watertight sealed access panel 13 through which components can be accessed.

The device 10 can include a processor 14, which can be a microcontroller having an embedded microprocessor, Random Access Memory (RAM) and Flash Memory (FM). FM can preferably contain the programming instructions for a control algorithm. FM can preferably be non-volatile and retains its programming when the power is terminated. RAM can be utilized by the control algorithm for storing variables such as pressure measurements, alarm counts and the like, which the control algorithm uses while generating and maintaining the vacuum for purposes of achieving one or more methodologies described herein, such as various permutations for continuous and intermittent compression and irrigation.

A membrane keypad and a light emitting diode LED or liquid crystal display (LCD) 16 can be electrically associated with processor 14 through a communication link, such as a cable. Keypad switches provide power control and are used to preset the desired pressure/vacuum levels. Light emitting diodes 17, 19 can be provided to indicate alarm conditions associated with canister fluid level, leaks of pressure in the dressing and canister, and power remaining in the power source.

Microcontroller 14 is electrically associated with, and controls the operation of, a first vacuum pump 18 and an optional second vacuum pump 20 through electrical connections. First vacuum pump 18 and optional second vacuum pump 20 can be one of many types including, for example, the pumps sold under the trademarks Hargraves® and Thomas®. Vacuum pumps 18 and 20 can use, for example, a reciprocating diaphragm or piston to create vacuum and can be typically powered by a direct current (DC) motor that can also optionally use a brushless commutator for increased reliability and longevity. Vacuum pumps 18 and 20 can be pneumatically associated with a disposable exudate collection canister 22 through a single-lumen tube 24.

In one embodiment, canister 22 has a volume which does not exceed 1000 ml. This can prevent accidental exsanguination of a patient in the event hemostasis has not yet been achieved at the wound site. Canister 22 can be of a custom design or one available off-the-shelf and sold under the trademark DeRoyal®.

In addition, a fluid barrier 26, which can be a back flow valve or filter, is associated with canister 22 and is configured to prevent fluids collected in canister 22 from escaping into tubing 24 and fouling the vacuum return path. Barrier 26 can be of a mechanical float design or may have one or more membranes of hydrophobic material such as those available under the trademark GoreTex™. Barrier 26 can also be fabricated from a porous polymer such as that which is available under the trademark MicroPore™. A secondary barrier 28 using a hydrophobic membrane or valve is inserted in-line with pneumatic tubing 24 to prevent fluid ingress into the system in the event barrier 26 fails to operate as intended. Pneumatic tubing 24 can connect to first vacuum pump 18 and optional second vacuum pump 20 through “T” connectors.

An identification member 30, such as radio frequency identification (RFID) tag, can be physically associated with the canister 22 and an RFID sensor 32 operably associated with the microcontroller 14 such that the microcontroller 14 can restrict use of the device 10 to a predetermined canister 22. Thus, if a canister 22 does not have a predetermined RFID chip, the device 10 will not operate. Another embodiment envisions software resident on microcontroller 14 which restricts the use of the device 10 to a predetermined time period such as 90 days for example. In this way, the patient using the device 10 may use the device 10 for a prescribed time period and then the device 10 automatically times out per a particular therapeutic plan for that patient. This also enables a reminder of the time and date for the next dressing change or physician appointment. It is also contemplated that the microcontroller 14 be operably provided with a remote control 15 and communication link, such as a transceiver, wherein the device 10 can be shut down remotely when a particular therapeutic plan for that patient has ended. Likewise, remote control 15 can be utilized to provide additional time after the therapeutic device times out.

Vacuum-pressure sensor 34 is pneumatically associated with first vacuum pump 18 and optional vacuum pump 20 and electrically associated with microcontroller 14. Pressure sensor 34 provides a vacuum-pressure signal to the microprocessor enabling a control algorithm to monitor vacuum pressure at the outlet of the vacuum pumps 18 and 20.

An acoustic muffler can be provided and pneumatically associated with the exhaust ports of vacuum pumps 18 and 20 and configured to reduce exhaust noise produced by the pumps during operation. In normal operation of device 10, first vacuum pump 18 can be used to generate the initial or “draw-down” vacuum while optional second vacuum pump 20 can be used to maintain a desired vacuum within the system compensating for any leaks or pressure fluctuations. Vacuum pump 20 can be smaller and quieter than vacuum pump 18 providing a means to maintain desired pressure without disturbing the patient. It is contemplated by the instant invention that pumps 18 and 20 can also be employed to create a positive pressure for purposes of applying pressure to an inflatable member 35, such as a cuff or pressure bandage, through tubing 36. A switch 37 can be operatively disposed on housing 12 in operable connection with microcontroller 14 to enable selection of positive and negative pressure from pumps 18/20.

One or more battery (ies) 38 can preferably be provided to permit portable operation of the device 10. Battery 38 can be Lithium Ion (LiIon), Nickel-Metal-Hydride (NiMH), Nickel-Cadmium, (NiCd) or their equivalent, and can be electrically associated with microcontroller 14 through electrical connections. Battery 38 can be of a rechargeable type which is preferably removably disposed in connection with the housing 12 and can be replaced with a secondary battery 38 when needed. A recharger 40 is provided to keep one battery 38 charged at all times. Additionally, it is contemplated that the device 10 can be equipped to be powered or charged by recharger 40 or by circuits related with microcontroller 14 if such source of power is available. When an external source of power is not available and the device 10 is to operate in a portable mode, battery 38 supplies power to the device 10. The battery 38 can be rechargeable or reprocessable and can preferably be removably stored in a waterproof manner within housing 12 which also likewise contains the pumps 18, 20 and microcontroller 14.

A second pressure sensor 42 is pneumatically associated with canister 22 through a sensor port 43. Pressure sensor 42 can be electrically associated with microcontroller 14 and provides a vacuum-pressure signal to microprocessor enabling control algorithm to monitor vacuum pressure inside canister 22 and dressing 11. A “T” connector can be connected to port 43, to pressure sensor 42 and a vacuum-pressure relief solenoid 46 configured to relieve pressure in the canister 22 and dressing 11 in the event of an alarm condition, or if power is turned off. Solenoid 46, can be, for example, one available under the trademark Parker Hannifin® or Pneutronics®; Solenoid 46 is electrically associated with, and controlled by, microprocessor of microcontroller 14. Solenoid 46 can be configured to vent vacuum pressure to atmosphere when an electrical coil associated therewith is de-energized as would be the case if the power is turned off. An orifice restrictor 48 may optionally be provided in-line with solenoid 46 and pneumatic tube 44 to regulate the rate at which vacuum is relieved to atmospheric pressure when solenoid 46 is de-energized. Orifice restrictor 48 is, for example, available under the trademark AirLogic®. In this regard, the pressure and in turn compression or vacuum can be operated in a continuous or intermittent manner, where pressure level can be varied or maintained in either a mode.

A wound dressing 11 can preferably include a sterile porous substrate 50, which can be a polyurethane foam, polyvinyl alcohol foam, gauze, felt or other suitable material, a semi-permeable adhesive cover 52 such as that sold under the trademark DeRoyal® or Avery Denison®, an inlet port 56 and a suction port 54. Substrate 50 is configured to distribute vacuum pressure evenly throughout the entire wound bed and has mechanical properties suitable for promoting the formation of granular tissue and approximating the wound margins.

In addition, when vacuum is applied to dressing 11, substrate 50 creates micro- and macro-strain at the cellular level of the wound stimulating the production of various growth factors and other cytokines, and promoting cell proliferation. Dressing 11 is fluidically associated with canister 22 through single-lumen tube 44. The vacuum pressure in a cavity formed by substrate 50 of dressing 11 is largely the same as the vacuum pressure inside canister 22 minus the weight of any standing fluid inside tubing 44.

A fluid vessel 60, which can be a standard IV bag, contains medicinal fluids such as aqueous topical antibiotics, analgesics, physiologic bleaches, or isotonic saline. Fluid vessel 60 is removably connected to dressing 11 though port 56 and single-lumen tube 62.

An optional flow control device 64 can be placed in-line with tubing 62 to permit accurate regulation of the fluid flow from vessel 60 to dressing 11. In normal operation, continuous wound site irrigation is provided as treatment fluids move from vessel 60 through dressing 11 and into collection canister 22. This continuous irrigation keeps the wound clean and helps to manage infection. The control device 64 can be operably connected to the microcontroller 14. In this way, the microcontroller 14 can control irrigation flow from vessel 60 in either a continuous or intermittent manner. In addition, effluent produced at the wound site and collected by substrate 50 will be removed to canister 22 when the system is under vacuum.

The device 10 is particularly well suited for providing therapeutic wound irrigation and vacuum drainage and provides for a self-contained plastic housing configured to be worn around the waist or carried in a pouch over the shoulder for patients who are ambulatory, and hung from the footboard or headboard of a bed for patients who are non-ambulatory. Membrane keypad and display 16 is provided to enable the adjustment of therapeutic parameters and to turn the unit on and off.

Depressing the power button on membrane switch 16 will turn the power to device 10 on/off. While it is contemplated that the membrane switch 16 be equipped with keys to adjust therapeutic pressure up and down, the microcontroller 14 can preferably be equipped to control the pressure in accordance with sensed pressure and condition to maintain pressure in an operable range between −70 mmHg and −150 mmHg with a working range of between 0 and −500 mmHg, for example. Although these pressure settings are provided by way of example, they are not intended to be limiting because other pressures can be utilized for wound-type specific applications. The membrane 16 can also be equipped with LED 17 to indicate a leak alarm and/or LED 19 indicates a full-canister alarm. When either alarm condition is detected, these LEDs will light in conjunction with an audible chime which is also included in the device 10.

Housing 12 can incorporate a compartment configured in such a way as to receive and store fluid vessel 60, such as a standard IV bag 60 or can be externally coupled to thereto. IV bag 60 may contain an aqueous topical wound treatment fluid that is utilized by the device 60 to provide continuous irrigation. A belt clip can be provided for attaching to a patient's belt and an optional waist strap or shoulder strap is provided for patients who do not or cannot wear belts.

Canister 22 is provided for exudate collection and can preferably be configured as currently known in the field with a vacuum-sealing means and associated fluid barrier 26, vacuum sensor port 43 and associated protective hydrophobic filter, contact-clear translucent body, clear graduated measurement window, locking means and tubing connection means. Collection canister 22 typically has a volume less than 1000 ml to prevent accidental exsanguination of a patient if hemostasis is not achieved in the wound. Fluid barriers 26 can be, for example, those sold under the trademark MicroPore® or GoreTex® and ensure the contents of canister 22 do not inadvertently ingress into pumps 18, 20 of housing 12 and subsequently cause contamination of thereof.

Pressure sensor 42 enables microcontroller 14 to measure the pressure within the canister 22 as a proxy for the therapeutic vacuum pressure under the dressing 11. Optionally, tubing 62 can be multilumen tubing providing one conduit for the irrigation fluid to travel to dressing 11 and another conduit for the vacuum drainage. Thus, IV bag 60, tubing 62, dressing 11 and canister 22 provide a closed fluid pathway. In this embodiment, canister 22 would be single-use disposable and may be filled with a solidifying agent 23 to enable the contents to solidify prior to disposal. Solidifying agents are available, for example, under the trademark DeRoyal® and Isolyzer®. The solidifying agents prevent fluid from sloshing around inside the canister particularly when the patient is mobile, such as would be the case if the patient were travelling in a motor vehicle. In addition, solidifying agents are available with antimicrobials that can destroy pathogens and help prevent aerosolization of bacteria.

At the termination of optional multilumen tubing 62, there can be provided a self-adhesive dressing connector 57 for attaching the tubing to drape 52 with substantially air-tight seal. Dressing connector 11 can have an annular pressure-sensitive adhesive ring with a release liner that is removed prior to application. Port 56 can be formed as a port cut in drape 52 and dressing connector 57 would be positioned in alignment with said port. This enables irrigation fluid to both enter and leave the dressing through a single port. In an alternative embodiment, tube 62 can bifurcate at the terminus and connect to two dressing connectors 57 which allow the irrigation port to be physically separated from the vacuum drainage port thus forcing irrigation fluid to flow though the entire length of the dressing if it is so desired. Similarly, port 54 and connector 55 can be provided to connect optional multilumen tubing 44 to dressing 11. In this arrangement, the second lumen may be used to directly measure the pressure in dressing 11.

Fluid vessel 60 can be of the type which includes a self-sealing needle port situated on the superior aspect of the vessel 60 and a regulated drip port situated on the inferior aspect of the vessel. The needle port permits the introduction of a hypodermic needle for the administration of aqueous topical wound treatment fluids. These aqueous topical fluids can include one or more of the following preferred irrigation solutions, Lactoferrin, Xylitol, Dakins solution, Polyhexanide, and Hypochlorous acid,

Lactoferrin (LF), also known as lactotransferrin (LTF), is a globular multifunctional protein with antimicrobial activity (bacteriocide, fungicide) and is part of the innate defense, mainly at mucoses. Lactoferrin is found in milk and many mucosal secretions such as tears and saliva. Lactoferrin is also present in secondary granules of PMN and also is secreted by some acinar cells. Lactoferrin can be purified from milk or produced recombinantly. Human colostrum (“first milk”) has the highest concentration, followed by human milk, then cow milk (150 mg/l).

Lactoferrin's antimicrobial activity is due partly to its high affinity for Fe3+ (ferric state). LF proteolysis produces the small peptides lactoferricin and kaliocin-1 with antimicrobial activity. The combination of iron and lactoferrin in mucosal secretions modulates the ability and aggregation of pathogenic bacteria, and inhibits both bacteria and viruses from binding to host cells. It is also an antifungal agent. Lactoferrin receptors have been found on brush-border cells, PMN, monocytes, macrophages and activated lymphocytes.

In contrast, support for the use of LF in wound healing is provided by its reported ability to induce collagen-gel contraction. In an in-vitro model for the reorganization of the collagen matrix that accompanies wound healing in skin during the remodeling phase, LF reduced the surface area by 50 percent in six hours through motility of fibroblasts by inducing phosphorylation of the myosin light chain. It has been shown that LF activities are mediated through the LRP receptor on fibroblasts and involve phosphorylation of ERK1/2 and activation of MLC kinase. LF has also been shown to stimulate the expression of IL-18, which appears to play an important role in the early phases of wound repair. IL-18 could in turn lead to an increase in GM-CSF, which also appears to be involved early in the wound repair process. GM-CSF has been reported to act on macrophages to secrete essential tissue growth factors and on keratinocytes to synthesize collagen. Moreover, since it is generally accepted that bacterial infections inhibit wound healing, the anti-infective and immunomodulatory properties of rhLF could be seen as relevant to wound healing, especially in diabetic ulcers, a condition where infections are common.

Xylitol is a sugar alcohol sweetener used as a naturally occurring sugar substitute. It is found in the fibers of many fruits and vegetables, including various berries, corn husks, oats, and mushrooms. It can be extracted from corn fiber, birch, raspberries, plums, and corn. Xylitol is roughly as sweet as sucrose with only two-thirds the food energy. As with most sugar alcohols, initial consumption can result in bloating, diarrhea, and flatulence, although generally rather less so than other sugar alcohols like sorbitol.

There are several ingredients to consider in this context. With respect to connective tissue repair and wound healing, Xylitol is necessary for efficient synthesis of the polysaccharide that is central to collagen. In collagen, links that provide for its strength are composed of Xylitol bridges. When such synthesis is impaired, the result is connective tissue disease (“a heterogeneous group of disorders, some hereditary, others acquired”).

Because of its central role in connective tissue repair, Xylitol is included in various preparations on the market for promoting such repair. An American company makes a product with Xylitol for animals, especially horses suffering joint and ligament problems.

The relation of Xylitol to osteoarthritis therapy is actually quite strong. Xylitol is a component of chondroitin sulfate, which is well known as a supplement benefiting arthritis sufferers. Chondroitin is directly utilized in forming the core molecules of cartilage. Xylitol is the first sugar to be attached in forming the glycoprotein chains involving chondroitin.

Dakins Solution is a highly diluted, neutral antiseptic solution for cleansing wounds consisting of sodium hypochlorite (0.45% to 0.5%) and boric acid (4%). Its solvent action on dead cells hastens the separation of dead from living tissue. The solution is unstable and cannot be stored more than a few days. Dakins Solution was developed during World War I.

Dakins solution is typically prepared using the following materials:
Sodium hypochlorite solution 5.25% (Clorox® or similar household bleach—unscented bleach as ultra bleach products that are more concentrated and thicker not recommended.
Sodium bicarbonate (baking soda)
Clean tap water
Clean pan with lid
Sterile measuring cup and spoons wherein equipment is to be sterilized or sanitized using a dishwasher on highest setting for hot water and heat.
Sterile jar with sterile lid

Procedure for making the Solution:

1. Wash hands well with soap and water.

2. Gather supplies.

3. Measure out 32 ounces (4 cups) of tap water and pour into the clean pan.

4. Boil water for 15 minutes with the lid on covering the pan and then remove from heat.

5. Using a sterile measuring spoon, add ½ teaspoonful of baking soda to the boiled water.

6. A physician may prescribe one of several strengths of the solution and thus measure bleach according to the following chart and add to the water:

| | □ Full | □ ½ | □ ¼ | □ ⅛ |
|---|---|---|---|---|
| | Strength | Strength | Strength | Strength |
| Clorox | 3 oz. | 3 Tbsp + ½ tsp. | 1 Tbsp + 2 tsp. | 2½ tsp. |
| | (or 9.5 ml) | (or 48 ml) | (or 24 ml) | (or 14-12 ml) |
| Water | 32 oz. | 32 oz. | 32 oz. | 32 oz. |

7. Place the solution in a sterile jar and close it tightly with the sterile lid and cover the entire jar with aluminum foil to protect it from light.

Polyaminopropyl biguanide (PAPB), also polyhexamethylene biguanide (PHMB) Prontosan®, polyhexamethylene guanide or polyhexanide, provide a disinfectant and a preservative used for disinfection on skin and in cleaning solutions for contact lenses. It is a polymer or oligomer where biguanide functional groups are connected by hexyl hydrocarbon chains, with varying chain lengths. PAPB is specifically bactericidal at very low concentrations (10 mg/l) and is also fungicidal.

The bactericidal has a unique method of action whereby the polymer strands are incorporated into the bacterial cell wall, which disrupts the membrane and reduces its permeability, which has a lethal effect to bacteria. It is also known to bind to bacterial DNA, alter its transcription, and cause lethal DNA damage. It has very low toxicity to higher organisms such as human cells, which have more complex and protective membranes. PAPB is a mixture of molecules of various sizes; different-sized molecules have a synergistic effect.

Some organisms such as Pseudomonas aeruginosa are able to develop resistance to this disinfectant.

PHMB is active against gram-negative and gram-positive bacteria, fungi and yeast including MRSA, Pseudomanas aeruginosa, VRE etc. PHMB has been in general use for about 60 years with no evidence of resistance. It is used in cosmetics, contact lens solution, swimming pools etc.

PHMB acts by electrostatic interactions, this mechanism is based on the character of the molecule and the distribution of electrical charges. This interferes with the bacterial cell metabolism, by prohibiting the cell's ability to absorb any nutrients or dispose of waste products. This effectively kills the bacteria without damaging surrounding healthy cells. PHMB is not adsorbed by cells and tissue, nor absorbed by them, and therefore cannot interfere with the metabolism of the body.

Advantages of PHMB are as follows:
excellent skin tolerance
non toxic, non irritant
hypoallergenic
suitable for long term use, not absorbed
can be used up to 8 weeks (Prontosan 350 ml Solution & Gel)
no inhibition of granulation tissue unlike antiseptics

Hypochlorous acid (Vashe®) is a weak acid with the chemical formula HClO. In the swimming pool industry, hypochlorous acid is referred to as HOCl. It forms when chlorine dissolves in water. It cannot be isolated in pure form due to rapid equilibration with its precursor. HClO is used as a bleach, an oxidizer, a deodorant, and a disinfectant. It is also very effective as an antibiotic. Escherichia coli exposed to hypochlorous acid lose viability in less than 100 ms due to inactivation of many vital systems.

Hypochlorous acid has a reported LD50 of 0.0104 ppm-0.156 ppm and 2.6 ppm caused 100% growth inhibition in 5 minutes. However, it should be noted that the concentration required for bactericidal activity is also highly dependent on bacterial concentration.

Wound healing is the end result of a series of interrelated cellular processes initiated by humoral factors such as cytokine growth factors. These cellular processes are inhibited by a large tissue bacterial bioburden. The cytokines and growth factors are also degraded by bacteria. The level of tissue bacterial bioburden has been shown in multiple studies to be more than 10⁵ or at least 1×10⁶ bacteria per gram of tissue. Such high levels of tissue bacteria can be present without clinical signs of infection, and when present can deleteriously affect wound healing.

Attempts at controlling the tissue bacterial bioburden have been difficult. Systemically administered antibiotics do not effectively decrease the level of bacteria in a chronic granulating wound. Therefore, topical antimicrobials or temporary biologic dressings have been the methods of choice. Topical use of antibiotics that are used effectively systemically for purposes other than wound infection is discouraged because of an increased risk for developing allergies or the potential for bacteria to develop resistance to the drug. Antiseptics and nonantibiotic antimicrobials such as povidone-iodine, silver sulfadiazine, or mafenide acetate cream have been demonstrated to be cytotoxic to the cellular components of wound healing.

Stabilized hypochlorous acid prepared by the addition of sodium hypochlorite to a solution of sodium chloride in sterile water followed by addition of a solution of hydrochloric acid and maintained at a pH between 3.5 and 5 has been demonstrated to have excellent in vitro antibacterial properties. Its potential limitation is the requirement to maintain its narrow pH range in the clinical wound environment.

Alternatively, solutions of hypochlorites can be produced by electrolysis of an aqueous chloride solution. Chlorine gas is produced at the anode, while hydrogen forms at the cathode. Some of the chlorine gas produced will dissolve forming hypochlorite ions through the above reaction. The geometry of the cell is critical to ensure that as much of the chlorine as possible dissolves, rather than simply bubbling out of the cell.

At the anode: 2Cl—→Cl2 (g)+2e−

At the cathode: 2H++2e−→H2 (g)

It can be seen that over time, the electrolyte will become increasingly basic.

There are a number of potential hazards and challenges associated with this process. It should not be attempted by untrained persons.

The electrochemical environment of the cell is highly corrosive, particularly at the anode. Few materials are suitable as an anode electrolyte. Graphite can be used, but will degrade quickly (which also results in contamination of the cell with finely divided carbon particles). Graphite supported lead dioxide electrodes have been reported to be more effective.

If the reaction conditions are not controlled, the produced hypochlorite can react with the hydroxide ions to form chlorate ions. These can additionally be electrochemically oxidized to perchlorate ions (within the same cell).

Hypochlorite is a powerful oxidizing agent, and will attack the dyes used in pH paper and damage pH sensors, making measurement and control of the conditions difficult.

Hydrogen gas is highly flammable, and can form explosive mixtures with both air and chlorine over a wide range of concentrations. Chlorine gas is highly toxic and corrosive.

Other irrigation solutions can include topical anesthetic such as Lidocaine, antibiotics such as Bacitracin or Sulfamide-Acetate; physiologic bleach such as Clorpactin; and antiseptics such as Lavasept or Octenisept.

Regulated drip port permits fluid within vessel 60 to egress slowly and continuously into porous substrate 50 whereupon the therapeutic benefits can be imparted to the wound site. Single-lumen drainage tube 44 provides enough vacuum to keep the dressing 11 at sub-atmospheric pressure and to remove fluids, which include the irrigation fluid and wound exudates. With this modification, the need for an external fluid vessel and associated tubing and connectors can be eliminated making the dressing more user friendly for patient and clinician alike.

In typical clinical use of this alternate embodiment, dressing 11 is applied to the wound site by first cutting porous substrate 50 to fit the margins of the wound. Next, semi-permeable drape 52 is attached and sealed over the dressing and periwound. A hole approximately ⅜″ diameter can be made in drape 52 central to porous substrate 50. Fluid vessel 60 is attached by adhesive annular ring 57 with port 56 aligned with the hole previously cut in drape 52. Once the fluid vessel 60 is hermitically sealed to the drape 52, a properly prepared hypodermic needle is inserted in self-sealing needle port and fluid vessel 60 subsequently filled with the desired aqueous topical wound treatment solution.

For the majority of applications, the technique for providing therapeutic wound irrigation and vacuum drainage is illustrated. The single lumen drainage tube 44 is provided for the application of vacuum and removal of fluids from the wound site. Fluid vessel 60 can be situated outside and superior to semi-permeable substrate 50. An annular adhesive ring 57 is provided on port 56 for attachment of single-lumen irrigation tubing 62 to drape 52. Similarly, a needle port permits the introduction of a hypodermic needle for the administration of aqueous topical wound treatment fluids as described above, for example, a caregiver may want to add a topical antibiotic to a bag of isotonic saline. Adjustable optional flow control device 64 permits fluid within vessel 60 to egress slowly and continuously into porous substrate 50 through hole 56 in drape 52 whereupon the therapeutic benefits can be imparted to the wound site. Single-lumen drainage tube 44 provides enough vacuum to keep the dressing 11 at sub-atmospheric pressure and to remove fluids which include the irrigation fluid and wound exudates.

Because of the potential chemical interactions between the various materials used in the construction of dressing 11, attention must be paid to the types of aqueous topical wound fluids used to ensure compatibility.

There are several methods of administration of the irrigation fluid. These are as follows:

Method 1—Continuous Irrigation, Continuous NPWT:

In this method, the suction is set to operate continuously at a pre-determined pressure level while an irrigation solution, comprised of one or more of the above listed topical treatment solutions, is introduced continuously at a pre-determined rate. Typically, for this application, the irrigation fluid would be introduced at a rate between 20 and 100 ml per hour with a preferable rate of between 30 and 60 ml per hour. Because the irrigation fluid is being continuously introduced to the wound site, a high infusion rate would necessitate frequent canister and fluid reservoir changes. Thus, it is preferable to introduce the fluid slowly over a longer period of time. The above referenced introduction rates have been shown to be clinically effective.

Method 2—Continuous Irrigation, Intermittent NPWT:

In this method, the suction is set to operate intermittently at predetermined pressure and “on/off” cycle durations while an irrigation solution, comprised of one or more of the above listed topical treatment solutions, and is introduced continuously at a pre-determined rate. During the portion of the interval while the suction is “off”, it is preferable to operate the suction at a low level, for example, 25 mmHg to ensure that the dressing seal is maintained at all times.

Typically, for this application, the irrigation fluid would be introduced at a rate between 20 and 100 ml per hour with a preferable rate of between 30 and 60 ml per hour. Because the irrigation fluid is being continuously introduced to the wound site, a high infusion rate would necessitate frequent canister and fluid reservoir changes. Thus, it is preferable to introduce the fluid slowly over a longer period of time. The above referenced introduction rates have been shown to be clinically effective.

Method 3—Continuous NPWT with Intermittent Irrigation (Flush before and after Dressing Change):

In this method, the suction is set to operate continuously at a pre-determined pressure level while an irrigation solution, comprised of one or more of the above listed topical treatment solutions is introduced intermittently at a pre-determined rate and frequency.

Typically, for this application, the irrigation fluid would be introduced at a rate between 500 and 1000 ml per minute. This method is especially effective as a treatment given before and/or after each dressing change. Essentially, in this configuration, the irrigation fluid acts as a type of “flush” that is particularly effective in minimizing pain prior to removal of the dressing. If additional pain management is desired, Lidocaine may be added to the topical solutions referenced above or in the alternative isotonic saline, for example. Flushing the dressing just after a dressing change helps maintain a moist healing environment for the wound and helps to keep dressing pores open during therapy. The above referenced introduction rates have been shown to be clinically effective.

Method 4—Intermittent NPWT with Intermittent Irrigation (Flood and Dwell):

In this method, the suction is set to operate intermittently at predetermined pressure and “on/off” cycle durations while an irrigation solution, comprised of one or more of the above listed topical treatment solutions, and is introduced intermittently at a pre-determined rate and frequency. The frequency of irrigation fluid introduction is synchronized with the application and non-application of suction pressure.

Typically, for this application, the irrigation fluid is not introduced while the suction is being applied to the wound but only once the suction is terminated. The dressing is normally flooded with the topical solution and held in place for a predetermined dwell time. After this dwell time has elapsed, the suction is once again applied and the irrigation fluid is subsequently drained from the dressing under vacuum. With this approach, the time during which the topical agents are in contact with the wound site is maximized. Quantities of topical fluids introduced on each cycle may range between 50 ml and 250 ml, for example. With this technique, care must be taken to ensure that the fluid is sufficient to provide desired clinical results while not so great as to cause a potential dressing leak and undesired fluid egress.

The above described embodiments are set forth by way of example and are not limiting. It will be readily apparent that obvious modifications, derivations and variations can be made to the embodiments. For example, the vacuum pumps described having either a diaphragm or piston-type could also be one of a syringe based system, bellows, or even an oscillating linear pump. Accordingly, the claims appended hereto should be read in their full scope including any such modifications, derivations and variations.

What is claimed is:

1. A therapeutic device, which includes:
a fluid mover for one of raising, compressing, or transferring fluid;
a therapeutic member operably connected to said fluid mover and actuated thereby, said therapeutic member operably disposably used on a patient in a manner to deliver therapy to the patient as function of actuation of said fluid mover;
a controller operably associated with said fluid mover for controlling operation thereof in a manner to cause one of continuous and intermittent actuation of said fluid mover.

2. The therapeutic device of claim 1, which includes an irrigation fluid in fluid communication with said therapeutic member.

3. The therapeutic device of claim 2, wherein said controller is equipped to control said fluid mover in a manner to provide one of continuous irrigation with continuous compression, continuous irrigation with intermittent compression, intermittent irrigation with continuous compression and intermittent irrigation with intermittent compression.

4. The therapeutic device of claim 2, wherein said controller is equipped to restrict use of said fluid mover by the patient in accordance with a predetermined treatment plan or duration and render said pump inoperable.

5. The therapeutic device of claim 1, which includes a chargeable power source to supply power to said fluid mover and said controller.

6. The therapeutic device of claim 2, which includes a leak sensor operably connected to said fluid mover and said controller and to sense a leak in said device and send a signal to said controller whereby said controller controls said fluid mover as a function of said sensed signal.

7. The therapeutic device of claim 1, which includes a fluid blockage sensor operably connected to said fluid mover and said controller and to sense a fluid blockage in said device and send a signal to said controller whereby said controller controls said fluid mover as a function of said sensed signal.

8. The therapeutic device of claim 7, wherein said fluid blockage sensor includes a pressure sensor.

9. The therapeutic device of claim 2, which includes a fluid blockage sensor operably connected to said fluid mover and said controller and to sense a fluid blockage in said device and send a signal to said controller whereby said controller controls said fluid mover as a function of said sensed signal.

10. The therapeutic device of claim 9, wherein said fluid blockage sensor includes a pressure sensor.

11. The therapeutic device of claim 1, which includes a temperature sensor operably connected to said controller and to sense a temperature in said device and send a signal to said controller whereby said controller controls said fluid mover as a function of said sensed signal.

12. The therapeutic device of claim 2, which includes a temperature sensor operably connected to said controller and to sense a temperature in said device and send a signal to said controller whereby said controller controls said fluid mover as a function of said sensed signal.

13. The therapeutic device of claim 1, which includes a voltage sensor operably connected to said device and said controller and to sense a voltage in said device and send a signal to said controller whereby said controller controls said fluid mover as a function of said sensed signal.

14. The therapeutic device of claim 2, which includes a voltage sensor operably connected to said device and said controller and to sense a voltage in said device and send a signal to said controller whereby said controller controls said fluid mover as a function of said sensed signal.

15. The therapeutic device of claim 1, which includes a current sensor operably connected to said device and said controller and to sense current in said device and send a signal to said controller whereby said controller controls said fluid mover as a function of said sensed signal.

16. The therapeutic device of claim 2, which includes a current sensor operably connected to said device and said controller and to sense current in said device and send a signal to said controller whereby said controller controls said fluid mover as a function of said sensed signal.

17. The therapeutic device of claim 5, wherein said chargeable power source is removable.

18. The therapeutic device of claim 2, wherein said controller includes a timer for restricting said use as a function of a predetermined time.

19. The disposable therapeutic device of claim 2, which further includes an identification member such that said controller restricts use as a function of a said identification member.

20. The disposable therapeutic device of claim 2, which includes a remote control for remotely controlling said controller.

21. The disposable therapeutic device of claim 2, which further includes a disposable container removably operably interconnected to said fluid mover and to said therapeutic member to receive waste fluid therein as a result of actuation of said fluid mover.

22. The disposable therapeutic device of claim 1, which further includes a housing operably containing said controller and said fluid mover.

23. The disposable therapeutic device of claim 22, wherein said housing is further characterized to contain said controller and said fluid mover in a waterproof manner.

24. A method of providing therapeutic treatment, which includes the steps of:
employing a fluid mover for performing at least one of raising fluid from, compressing, or transferring fluid across a wound site through a therapeutic member operably connected to said fluid mover and which is actuated thereby, said therapeutic member operably disposably used on a patient in a manner to deliver therapy to the patient as function of actuation of said fluid mover, and using an irrigation fluid in fluid communication with said therapeutic member; and
controlling said fluid mover in a manner to cause one of continuous and intermittent actuation of said fluid mover.

25. The method of claim 24, which includes controlling irrigation in a manner to cause one of continuous and intermittent flow of said irrigation fluid.

26. The method of claim 25, which is further characterized to include continuous flow of said irrigation fluid and continuous compression.

27. The method of claim 25, wherein said irrigation fluid is a topical treatment irrigation solution.

28. The method of claim 26, wherein said fluid mover operates continuously at a pre-determined pressure level and said an irrigation solution is introduced continuously at a pre-determined rate.

29. The method of claim 25, which is further characterized to include continuous flow of said irrigation fluid with intermittent compression.

30. The method of claim 29, wherein said irrigation fluid is a topical treatment irrigation solution.

31. The method of claim 30, wherein said fluid mover operates intermittently at a pre-determined pressure level and said an irrigation solution is introduced continuously at a pre-determined rate.

32. The method of claim 25, which is further characterized to include intermittent flow of said irrigation fluid with intermittent compression.

33. The method of claim 32, wherein said irrigation fluid is a topical treatment irrigation solution.

34. The method of claim 33, wherein said fluid mover operates intermittently at a pre-determined pressure level and said an irrigation solution is introduced intermittently at a pre-determined rate.

35. The method of claim 25, which is further characterized to include intermittent flow of said irrigation fluid with continuous compression.

36. The method of claim 36, wherein said irrigation fluid is a topical treatment irrigation solution.

37. The method of claim 36, wherein said fluid mover operates continuously at a pre-determined pressure level and said an irrigation solution is introduced intermittently at a pre-determined rate.

38. The method of claim 24, wherein said topical treatment irrigation solution includes one or more of Lactoferrin, Xylitol, Dakins solution, Polyhexanide, and Hypochlorous acid, Lidocaine, Bacitracin, Sulfamide-Acetate, Chlorpactin, Lavasept or Octenisept.
